(19) United States
(12) Patent Application Publication
(10) Pub. No.: US 2007/0218535 A1
(43) Pub. Date: Sep. 20, 2007

(54) Methods for production of recombinant alpha1-antitrypsin

(76) Inventors: Xinli Lin, Irvine, CA (US); Daniel Medynski, Oakland, CA (US)

Correspondence Address: MORRISON & FOERSTER LLP, 755 PAGE MILL RD, PALO ALTO, CA, 94304-1018, US

(21) Appl. No.: 11/605,619

(22) Filed: Nov. 28, 2006

Related U.S. Application Data
(60) Provisional application No. 60/740,335, filed on Nov. 28, 2005.

(51) Int. Cl. C12N 9/64

(52) U.S. Cl. 435/226

(57) ABSTRACT

Methods of producing properly folded recombinant α1-antitrypsin (AAT) polypeptide are provided. Denatured recombinant AAT polypeptide is refolded by first solubilizing the polypeptide with a chaotroph at high pH, followed by refolding in the presence of reduced concentrations of chaotroph and in the presence of PEG, glycerol or sucrose, or a detergent while the pH is slowly reduced and is generally maintained.

### CROSS-REFERENCES TO RELATED APPLICATIONS

This application claims priority benefit of provisional patent application Ser. No. 60/740,335, filed Nov. 28, 2005, which is incorporated in its entirety by reference.

### STATEMENT REGARDING FEDERALLY SPONSORED RESEARCH OR DEVELOPMENT

Not applicable.

### FIELD OF THE INVENTION

This invention relates to methods for producing recombinant α1-antitrypsin (AAT) polypeptides.

### BACKGROUND OF THE INVENTION

Neutrophils can pass through the capillaries of the lung unobstructed and are important mediators of immune response against invading microbial pathogens in the lung. Neutrophils release a wide range of defensive molecules as part of an inflammatory response, including reactive molecular oxygen species, cationic peptides, eicosanoids, and proteolytic enzymes. Lee et al., Am. J. Perspir. Crit. Care Med. 164:896-904, 2001. Paradoxically, unregulated release of these molecules can lead to serious lung damage.

Human leukocyte elastase (HLE), also known as human neutrophil elastase (HNE), is a serine protease released from the azurophilic granules of the neutrophil as part of the normal inflammatory response. Shapiro, Eur. Respir. J. Suppl. 44:30s-32s, 2003. Under normal homeostatic conditions, α1-antitrypsin (AAT; A1AT) serves as an important regulator of proteolysis by HLE, thereby preventing damage of the lung alveolar matrix. AAT is a 52 Kda glycoprotein synthesized primarily in the liver, but also in neutrophils, monocytes, and macrophages. AAT is secreted into the blood plasma, but its primary site of action is in the lung parenchyma. Moraga et al., J. Biol. Chem. 275:7693-7000, 2000. Besides HLE, AAT also inhibits two other proteases released into the lungs by neutrophils, namely cathepsin G (catG) and protease 3 (Pr3). CatG and Pr3 may also contribute to lung damage by breaking down elastin biters and other extracellular matrix proteins. AAT may prevent this damage. However, HLE is considered to be the enzyme primarily responsible for lung damage. Korkmaz et al., Amer. J. Resp. Cell Mol. Biol. 32:553-559, 2005.

Inherited deficiency of AAT predisposes individuals to early onset hereditary emphysema, due to the unregulated action of HLE and possibly other proteases. Crystal et al., Hosp. Pract. (Off Ed). 26(2):81-4, 88-9, 93-4; 1991. Large quantities and frequent injections of AAT are required to restore normalcy to the lungs and relieve hereditary emphysema. Presently, AAT supplies are isolated from pooled blood products (e.g. Zemaira®, Prolastin®), and there is a greater demand for the product relative to the available supply. Because of the limited supply of AAT, it has not been adequately tested for its beneficial effects in other respiratory disorders. AAT may also be useful in the treatment of emphysema caused by smoking, cystic fibrosis, pulmonary hypertension, pulmonary fibrosis, and chronic obstructive pulmonary disease (COPD). Cantin et al., J. Aerosol. Med. 15: 141-148, 2002; Cowan et al., Nat. Med. 6:698-702, 2000; Obayashi et al. Chest 112: 1338-1343, 1997.

Human AAT is a 394 amino protein that has been cloned and expressed in heterologous expression systems including E. coli and S. cerevisiae. Bollen et al., DNA 2:255-264, 1983; Kurachi et al., Proc. Natl. Acad. Sci. U.S.A. 78:6826-6830, 1981; Johansen et al., Mol. Biol. Med. 4:291-305, 1987; Kwon et al., Biochimica et Biophysica Acta 1247:179-184, 1995. It has been reported that deletion of the first 5 or 10 amino acids in human AAT leads to high level production in E. coli, and the produced AAT derivatives with N-terminal truncations exhibit specific activities in trypsin and elastase inhibition assays identical to authentic human AAT. Johansen et al., Mol. Biol. Med. 4:291-305, 1987. Human AAT is expressed as a 418 amino acid precursor from which a 24 amino acid precursor is clipped to yield the 394 amino acid final product. The native product has 3 glycosylation sites. The crystal structure of AAT has been solved at 2.0 angstroms and intensively studied, both as a native molecule, and in some of the disease causing variant forms. Elliott et al., Protein Sci. 9:1274-1281, 2000.

Because prokaryotes such as E. coli lack the biochemical machinery necessary to glycosylate proteins, AAT produced in E. coli is not glycosylated. It has been demonstrated that the in vivo half life of non-glycosylated AAT is at least 6 fold shorter than glycosylated AAT, limiting its therapeutic effectiveness. Weber et al., Biochem. Biophys. Res. Commun. 126(1):630-5, 1985. It has been reported that in vivo half life of many biological molecules can be prolonged by pegylation of the molecules, and polyethylene glycol conjugation at Cys²³² prolongs the half-life of AAT. Cantin et al., Am. J Respir. Cell Mol. Biol. 27:659-665, 2002; Graddis et al., Curr. Pharm. Biotechnol. 3(4):285-97, 2002; Molineux, Cancer Treat. Rev. 28 Suppl A:13-6, 2002.

Methods for producing refolded polypeptides have been reported in U.S. Pat. Nos. 6,583,268, and 7,119,166; U.S. Pub. Nos. 2003/0199676, 2004/0265298, 2005/0227920; PCT WO 01/55174, WO 2004/094344, WO 2005/058930.

All patents, patent applications, and publications cited herein are hereby incorporated by reference in their entirety.

### BRIEF SUMMARY OF THE INVENTION

The invention provides a new refolding method to produce α1-antitrypsin (AAT) polypeptides in active form. The instant methods utilize denatured AAT polypeptides, and generate correctly folded, highly active AAT polypeptides using only a small number of steps. In some embodiments, the methods utilize crude bacterially-produced AAT polypeptide (e.g., either from cell paste or inclusion bodies), and generate correctly folded, highly active AAT polypeptides.

In one aspect, the invention provides a method for producing a refolded recombinant AAT polypeptide comprising: a) solubilizing a denatured AAT polypeptide with a solubilization buffer comprising a high concentration of chaotroph, a reducing agent, and having a pH of about 8.5 to about 11.0, to produce a solubilized AAT polypeptide solution; b) diluting the solubilized AAT polypeptide solution with a refolding buffer by adding the solubilized AAT polypeptide solution into the refolding buffer to produce a diluted solubilized AAT polypeptide solution, wherein the refolding buffer comprises glycerol, a sugar, or polyethylene glycol (PEG), or any combination thereof; and c) reducing the pH of the diluted solubilized AAT polypeptide solution to a pH of about 7.5 to about 8.5, wherein said pH reducing is carried out over a period of at least about 20 hours, thereby producing a refolded AAT polypeptide.

In some embodiments, the chaotroph is urea, which may be at about 8 M concentration. In other embodiments, the chaotroph is guanidine hydrochloride, which may be at about 6 M concentration.

In some embodiments, the solubilized AAT polypeptide solution is diluted about twenty-fold into the refolding buffer.

In some embodiments, the refolding buffer comprises Tris as the buffer. In some embodiments, the refolding buffer comprises glycerol, sucrose, or any combination thereof For example, the refolding buffer may comprise about 5% to about 30% glycerol, about 10% to about 30% sucrose, or about 10% glycerol and about 10% sucrose. In some embodiments, the refolding buffer comprises PEG. In some embodiments, molecular weight of the PEG is about 200 to about 20,000 Daltons. In some embodiments, the molecular weight of the PEG is about 200 Daltons. In some embodiments, the molecular weight of the PEG is about 600 Daltons. In some embodiments, the refolding buffer may further comprises a detergent, such as Tween 20, Tween 80, sodium deoxycholate, sodium cholate, and trimethylamine-N-oxide (TMSO).

In some embodiments, the solubilization buffer or the refolding buffer is about pH 8.5 to about pH 10.8. In some embodiments, the solubilization buffer or the refolding buffer is about pH 10.0 to about pH 10.8. In some embodiments, the solubilization buffer and/or the refolding buffer is about pH 8.5, about pH 9.0, about pH 10, about pH 10.5, or about pH 10.8. In some embodiments, the solubilization buffer and the refolding buffer have the same pH.

In some embodiments, the pH of the diluted solubilized AAT polypeptide solution is reduced to about pH 8.0.

In some embodiments, the method further comprises adjusting the A₂₈₀ of the solubilized AAT polypeptide solution to about 2.0 to about 10.0 (e.g., about 2.0 to about 5.0) with a solubilization buffer before diluting the solubilized AAT polypeptide solution with the refolding buffer.

In an exemplary embodiment, the method for producing a refolded recombinant AAT polypeptide comprises: a) solubilizing a denatured AAT polypeptide with a solubilization buffer comprising about 8 M urea, about 0.1 M Tris, about 1 mM glycine, about 1 mM EDTA, about 100 mM β-mercaptoethanol, at about pH 10.5, to produce the solubilized AAT polypeptide solution; b) adjusting A₂₈₀ of the solubilized AAT polypeptide solution to 2.0 with a solubilization buffer comprising about 8 M urea, about 0.1 M Tris, about 1 mM glycine, about 1 mM EDTA, about 10 mM β-mercaptoethanol, about 10 mM dithiothreitol (DTT), about 1 mM reduced glutathione (GSH), at about pH 10.5; c) rapidly diluting the solubilized AAT polypeptide solution with the refolding buffer by adding the solubilized AAT polypeptide solution into about twenty volumes of the refolding buffer comprising about 20 mM Tris, pH about 10.5, and any of 1) about 10% to about 30% glycerol, 2) about 10 to about 30% sucrose, 3) about 20% glycerol and about 20% sucrose, 4) about 10% glycerol and about 10% sucrose, and 5) about 5% to about 10% PEG; and d) reducing the pH of the diluted solubilized AAT polypeptide solution to a pH of about 7.6 over a period of at least about 20 hours to 4 days, thereby producing the refolded AAT polypeptide. In some embodiments, the refolding buffer further comprises about 0.005% to about 0.02% Tween 20.

In another exemplary embodiment, the method for producing a refolded recombinant AAT polypeptide, comprises: a) solubilizing a denatured AAT polypeptide with the solubilization buffer comprising about 8 M urea, about 0.1 M Tris, about 1 mM glycine, about 1 mM EDTA, about 10 mM β-mercaptoethanol, about 10 mM dithiothreitol (DTT), about 1 mM reduced glutathione (GSH), at about pH 10.5, to produce the solubilized AAT polypeptide solution; b) rapidly diluting the solubilized AAT polypeptide solution with a refolding buffer by adding the solubilized AAT polypeptide solution into about twenty volumes of the refolding buffer comprising about 20 mM Tris and about 20% sucrose, pH about 10.5; and c) reducing the pH of the diluted solubilized AAT polypeptide solution to a pH of about 7.6 over a period of at least about 20 hours, thereby producing the refolded AAT polypeptide.

In another aspect, the invention also provides a method for producing a refolded recombinant AAT polypeptide comprising: a) solubilizing a denatured AAT polypeptide with a solubilization buffer comprising a high concentration of chaotroph, a reducing agent, and having a pH of about 8.5 to about 10.5, to produce a solubilized AAT polypeptide solution; b) diluting the solubilized AAT polypeptide solution with a refolding buffer having a pH of about 8.5 to about 10.5 by adding the solubilized AAT polypeptide solution into the refolding buffer to produce a diluted solubilized AAT polypeptide solution, wherein the refolding buffer comprises glycerol, a sugar, or PEG, or any combination thereof; c) incubating the diluted solubilized AAT polypeptide solution for at least about 16 hours at a temperature of about 16° C. to about 20° C.; d) further incubating the diluted solubilized AAT polypeptide solution at about 4° C. for about 24 to about 72 hours; and e) exchanging the diluted solubilized AAT polypeptide solution to a buffer having a pH of about 7.5 to about 8.5, thereby producing a refolded AAT polypeptide. In some embodiments, the buffer in step e) has the same formulation as the refolding buffer.

In some embodiments, the method further comprises a step of concentrating the diluted solubilized AAT polypeptide solution before step e). For example, ultrafiltration concentration may be used to concentrate the AAT polypeptide to about 20-200 fold. In some embodiments, step e) is performed by dialysis or size exclusion chromatography.

In some embodiments, the chaotroph is urea, which may be at about 8 M concentration. In other embodiments, the chaotroph is guanidine hydrochloride, which may be at about 6 M concentration.

In some embodiments, the solubilized AAT polypeptide solution is diluted about twenty-fold into the refolding buffer.

In some embodiments, the refolding buffer comprises Tris as the buffer. In some embodiments, the refolding buffer comprises glycerol, sucrose, or any combination thereof. For example, the refolding buffer may comprise about 5% to about 20% glycerol, about 10% to about 20% sucrose, or about 10% glycerol and about 10% sucrose. In some embodiments, the refolding buffer comprises PEG. In some embodiments, molecular weight of the PEG is about 200 to about 20,000 Daltons. In some embodiments, the molecular weight of the PEG is about 200 Daltons. In some embodiments, the molecular weight of the PEG is about 600 Daltons. In some embodiments, the refolding buffer may further comprises a detergent, such as Tween 20, Tween 80, sodium deoxycholate, sodium cholate, and trimethylamine-N-oxide (TMSO).

In some embodiments, the solubilization buffer or the refolding buffer is about pH 8.5. In some embodiments, the solubilization buffer and/or the refolding buffer is about pH 9.0. In some embodiments, the solubilization buffer and/or the refolding buffer is about pH 9.5. In some embodiments, the solubilization buffer and/or the refolding buffer is about pH 10.0. In some embodiments, the solubilization buffer and the refolding buffer have the same pH.

In some embodiments, the method further comprises adjusting the A₂₈₀ of the solubilized AAT polypeptide solution to about 2.0 to about 10.0 (e.g., about 2.0 to about 5.0) with a solubilization buffer before diluting the solubilized AAT polypeptide solution with the refolding buffer.

In an exemplary embodiment, the method for producing a refolded recombinant AAT polypeptide comprises: a) solubilizing a denatured AAT polypeptide with a solubilization buffer comprising about 8 M urea, about 0.1 M Tris, about 1 mM glycine, about 1 mM EDTA, about 100 mM β-mercaptoethanol, at about pH 10.5, to produce the solubilized AAT polypeptide solution; b) adjusting A₂₈₀ of the solubilized AAT polypeptide solution to 2.0 with a solubilization buffer comprising about 8 M urea, about 0.1 M Tris, about 1 mM glycine, about 1 mM EDTA, about 10 mM β-mercaptoethanol, about 10 mM dithiothreitol (DTT), about 1 mM reduced glutathione (GSH), at about pH 10.5; c) rapidly diluting the solubilized AAT polypeptide solution with a refolding buffer by adding the solubilized AAT polypeptide solution into about twenty volumes of the refolding buffer comprising about 20 mM Tris, pH 8.5, and any of 1) about 10% glycerol, 2) about 10% to about 30% sucrose, 3) about 10% glycerol and about 10% sucrose, and 4) about 5% to about 10% PEG; d) incubating the diluted solubilized AAT polypeptide solution for at least about 16 hours at about 20° C.; e) further incubating the diluted solubilized AAT polypeptide solution at about 4° C. for about 24 to about 72 hours; f) concentrating the diluted solubilized AAT polypeptide solution by ultrafiltration; and g) exchanging the diluted solubilized AAT polypeptide solution to a buffer comprising about 20 mM Tris, about 0.2 M NaCl, about 10% glycerol or about 15% sucrose, about 1 mM DTT, pH 7.6 by size exclusion chromatography, thereby producing a refolded AAT polypeptide. In some embodiments, the refolding buffer and the buffer in step g) further comprises about 0.005% Tween 20.

The following embodiments are generally applicable for any of the methods described herein.

In some embodiments, the AAT polypeptide is a human AAT polypeptide (SEQ ID NO:1). In some embodiments, one or more amino acid residues within amino acids 1-15 of SEQ ID NO:1 are deleted. For AAT polypeptide with N-terminal deletions, a methionine may be added as the first codon to enable expression in E. coli. In some embodiments, the AAT polypeptide comprises amino acids 2-394, 3-394, 4-394, 5-394, 6-394, 7-394, 8-394, 9-394, 10-394, or 11-394 of SEQ ID NO:1. In some embodiments, the AAT polypeptide comprises the sequence of SEQ ID NO:3.

In some embodiments, the method further comprises a step of conjugating a polyethylene glycol (PEG) molecule to the AAT polypeptide. In some embodiments, the PEG molecule is conjugated to amino acid Cys²³² of the AAT polypeptide, wherein the amino acid numbering for Cys²³² is based on amino acid numbering in SEQ ID NO:1. In some embodiments, the PEG molecule has a molecular weight of about 20 kD to about 40 kD.

The methods of the invention may comprise additional steps at the beginning of the process. Thus, in some embodiments the method includes the preliminary step of lysing bacterial host cells comprising denatured AAT polypeptide and collecting said denatured AAT polypeptide. Certain additional embodiments also include washing the denatured AAT polypeptide.

The methods of invention may also comprise additional steps at the end of the process. Thus, some embodiments also include purification of the refolded AAT polypeptide, such as by size exclusion chromatography (SEC), anion exchange chromatography, a hydrophobic interaction chromatography, or any combination of these steps, such as SEC followed by anion exchange chromatography, and further followed by hydrophobic interaction chromatography, which can be used in any order.

The invention also provides a method for purification of a properly folded AAT polypeptide from improperly folded or unfolded AAT comprising a) binding of the improperly folded or unfolded AAT polypeptide to a hydrophobic interaction chromatography resin in the presence of a salt; and b) collecting the properly folded AAT polypeptide which is not bound to the resin. In some embodiments, the salt is (NH₄)₂SO₄ or NaCl. In some embodiments, about 0.25 to about 1.2 M (NH₄)2SO₄ is used. In some embodiments, about 1.5 M to about 3.5 M NaCl is used. In some embodiments, the properly folded AAT polypeptide is from bacterial inclusion bodies.

The invention also provides AAT polypeptides produced by the instant methods. In some embodiments, the AAT polypeptide is unglycosylated AAT polypeptide. In some embodiments, the AAT polypeptide produced by the instant methods has a purity of at least about any of 80%, 85%, 90%, 95%, 96%, 97%, 98%, and 99%.

The invention also provides pharmaceutical compositions comprising an AAT polypeptide described herein and a pharmaceutically acceptable excipient. The invention also provides kits comprising an AAT polypeptide described herein. These kits, generally in suitable packaging and provided with appropriate instructions, are useful for treating an individual with AAT deficiency (including hereditary and nonhereditary).

### BRIEF DESCRIPTION OF THE SEVERAL VIEWS OF THE DRAWING(S)

FIG. 1 shows the amino acid sequence (SEQ ID NO:1) of the mature form of native human AAT and the cDNA sequence (SEQ ID NO:2) encoding the mature form of native human AAT.

FIG. 2 shows the synthetic cDNA sequence (SEQ ID NO:4) and translated protein sequence (SEQ ID NO:3) optimized for expression of a truncated AAT polypeptide (Δ5 AAT) in E. coli. This truncated AAT polypeptide, derived from human mature AAT sequence, lacks amino acids 1-5 but possesses an artificial methionine which enables the initiation of protein expression.

FIG. 3 shows a non-reducing SDS PAGE demonstrating that unpegylated and pegylated Δ10 AAT polypeptide (lanes 2 and 3 respectively) and Δ5 AAT polypeptide (lanes 4 and 5 respectively) produced from E. coli could be purified to near homogeneity. Molecular weight markers are indicated for each SDS PAGE (lanes 1 and 6).

FIG. 4 shows comparison of inhibitory activity of the refolded and purified pegylated and unpegylated Δ5 AAT polypeptide in comparison to commercial full length human AAT (glycosylated) in blocking enzymatic activity of HLE and PPE.

FIG. 5 shows SDS-PAGE and MALDI-TOF mass spectrometry of pegylated Δ5 AAT polypeptide. FIG. 5A shows an SDS-PAGE gel of unpegylated and pegylated AAT. Lane 1: unpegylated Δ5 AAT polypeptide; lane 2: pegylated Δ5 AAT polypeptide; and lane 3: molecular weight markers. FIG. 5B shows MALDI-TOF mass spectrometry of a sample after pegylation reaction. The molecular weight of each of the indicated peaks is depicted at the top of the respective peak. For instance, the molecular weight of the Δ5 AAT polypeptide (unpegylated) is 43996.34 daltons, while the molecular weight of the pegylation reagent Mal-PEG 20 is 22063.92 daltons. The successfully pegylated Δ5 AAT has a molecular weight of 65324.02 daltons, in close agreement with its predicted molecular weight. This indicates that the Δ5 AAT has been successfully pegylated.

FIG. 6 shows SDS-PAGE of fractions of AAT polypeptide collected from hydrophobic interaction chromatography. Lanes 1-6 show non-reduced SDS-PAGE; and lane 7 shows reduced SDS-PAGE. Lane 1 shows molecular weight marker. Lane 2 shows fractions containing AAT polypeptide flowed through the hydrophobic interaction column in the presence of 1 M (NH₄)₂SO₄. Lanes 3-6 show factions containing AAT polypeptide eluted from the column when the (NH₄)₂SO₄ is removed from the buffer. Lane 7 shows the same factions as lane 2 run on reduced SDS-PAGE.

FIG. 7 shows the comparison of inhibitory activity of refolded and purified Δ5 AAT polypeptide with Zemaria® in inhibiting PPE.

### DETAILED DESCRIPTION OF THE INVENTION

The instant invention provides methods for the production of recombinant, biologically active AAT polypeptides.

The practice of the present invention will employ, unless otherwise indicated, conventional techniques of immunology, molecular biology, microbiology, cell biology and recombinant DNA, which are within the skill of the art. See, e.g., Molecular Cloning: a laboratory manual, 2^{nd} edition Sambrook, et al. (1989); Current Protocols In Molecular Biology F. M. Ausubel, et al. eds., (1987); the series Methods In Enzymology, Academic Press, Inc.; PCR 2: A Practical Approach, M. J. MacPherson, B. D. Hames and G. R. Taylor, eds. (1995), and Antibodies, A Laboratory Manual, Harlow and Lane, eds. (1988).

It should be noted that, as used herein, the singular form “a”, “an”, and “the” includes plural references unless indicated otherwise.

When “about” is used to describe a range, the term applies to both lower and upper value of a range. For example, “about X to Y” means “about X to about Y”.

It is understood that aspect and embodiments of the invention described herein include “consisting” and/or “consisting essentially of” aspects and embodiments.

A. AAT Polypeptides

AAT polypeptide (used interchangeably with AAT, AAT protein) includes any naturally occurring species (such as full length from any mammalian, e.g., human, farm animals, sport animals, pets, primates, horses, dogs, cats, mice and rats), biologically active polypeptide fragments (such as fragment from human AAT with one or more amino acid deletion at the N-terminal of the protein), and variants (including naturally occurring and non-naturally occurring), including functionally equivalent variants which do not significantly affect their biological properties and variants which have enhanced or decreased activity (e.g., inhibition activity to human leukocyte elastase). Examples of variants include AAT with one or more amino acid substitution (e.g., conservative substitution), one or more deletions or additions of amino acids which do not significantly change the folding and/or functional activity of the protein.

AAT polypeptides, including variants, peptide fragments, modified forms of AAT polypeptides (including naturally occurring AAT), fusion protein and conjugate of the invention, may be characterized by any one or more of the following characteristics: (a) ability to inhibit proteolysis activity of leukocyte elastase (e.g., human leukocyte elastase (HLE)); (b) ability to inhibit proteolysis activity of porcine pancreatic elastase; (c) ability to inhibit proteolysis activity of cathepsin G (catG) (e.g., human catG); (d) ability to inhibit proteolysis activity of proteinase 3 (e.g., human Pr3); and (e) prevent HLE mediated lung injury. Thus all AAT polypeptides (including variants, fragments, and modified forms) are functional as described above. The inhibition may be complete or partial, e.g., at least about 50%, at least about 60%, at least about 70%, at least about 80%, at least about 90%, at least about 95%, or 100% of the enzymatic activity is inhibited.

In some embodiments, the AAT polypeptide comprises amino acid sequence of SEQ ID NO:1. In some embodiments, the AAT polypeptide comprises a deletion of one or more amino acid residues within amino acids 1-15 of SEQ ID NO:1. In some embodiments, the AAT polypeptide comprises various amino-terminal truncated AAT fragment. For example, the AAT polypeptide comprises amino acids 2-394, 3-394, 4-394, 5-394, 6-394, 7-394, 8-394, 9-394, 10-394, or 11-394 of SEQ ID NO:1. The amino terminal truncated AAT fragment may have a methionine added at the N-terminus to enable polypeptide expression, for example, in E. coli. In some embodiments, the AAT comprises amino acid sequence of SEQ ID NO:3 or amino acid sequence encoded by nucleic acid sequence of SEQ ID NO:4. AAT polypeptide embodiments include fusion proteins (N-terminal fusion or C-terminal fusion).

Variants of AAT of the present invention may include one or more amino acid substitutions, deletions or additions that do not significantly change the activity of the protein. Variants may be from natural mutations or human manipulation. Changes can be of a minor nature, such as conservative amino acid substitutions that do not significantly affect the folding or activity of the protein. To improve or alter the characteristics of AAT polypeptides, protein engineering may be employed. Recombinant DNA technology known to those skilled in the art can be used to create novel mutant proteins or mutants including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Thus, AAT also encompasses AAT derivatives and analogs that have one or more amino acid residues deleted, added, or substituted to generate AAT polypeptides that are better suited for expression, scale up, etc., in the host cells chosen. In some embodiments, amino acid sequences of the AAT variants are at least about any of 70%, 75%, 80%, 85%, 90%, 95%, 96%, 97%, 98%, or 99% identical to a AAT (such as from a mammalian, a human AAT). Examples of variant forms of AAT are described in U.S. Pat. Nos. 4,732,973, 5,134,119, and 4,711,848, which are incorporated herein by reference in their entirety.

Two polypeptide sequences are said to be “identical” if the sequence of amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e. the window size) and multiplying the results by I 00 to yield the percentage of sequence identity.

Variants of AAT polypeptide also encompass fusion proteins comprising AAT polypeptides. Biologically active AAT polypeptides can be fused with sequences, such as sequences that facilitate the coupling of the polypeptide to a support or a carrier, or facilitate refolding and/or purification (e.g., sequences encoding epitopes such as Myc, HA derived from influenza virus hemagglutinin, His-6, FLAG, or His-Tag). These sequences may be fused to AAT polypeptide at the N-terminal end or at the C-terminal end. In addition, the protein or polynucleotide can be fused to other or polypeptides which increase its function, or specify its localization in the cell, such as a secretion sequence. Methods for producing recombinant fusion proteins described above are known in the art. The recombinant fusion protein can be produced, refolded, and isolated by methods described herein and other methods known in the art.

Variants of AAT polypeptide also include conjugate comprising any of the AAT polypeptide embodiments described herein, e.g., an AAT polypeptide conjugated or fused to a half life extending moiety, such as a PEG or a peptide.

Variants of AAT also include functional equivalent variants. Functional equivalent variants are identified any one or more of the following criteria:(a) ability to inhibit proteolysis activity of leukocyte elastase (e.g., human leukocyte elastase (HLE)); (b) ability to inhibit proteolysis activity of porcine pancreatic elastase; (c) ability to inhibit proteolysis activity of cathepsin G (catG) (e.g., human catG); (d) ability to inhibit proteolysis activity of proteinase 3 (e.g., human Pr3); and (e) prevent HLE mediated lung injury. Biological activity of variants of AAT polypeptide may be tested using methods known in the art and methods described herein. In some embodiments, functional equivalent variants have at least about any of 50%, 60%, 70%, 75%, 80%, 85%, 90%, or 95% of activity as compared to full length native AAT with respect to one or more of the biological assays described above (or known in the art).

B. Methods of Refolding AAT Polypeptides

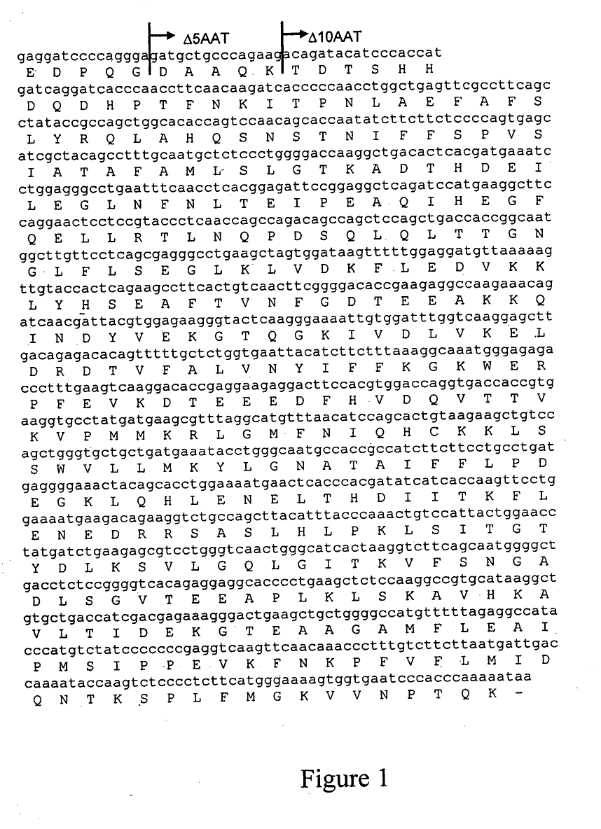

The methods of the invention are typically practiced utilizing inclusion bodies containing AAT polypeptide, such as those formed in bacterial (e.g., E. coli) cells which have been engineered to produce AAT (such as human AAT polypeptide), as the starting material, but any source of denatured AAT polypeptide may be used. The AAT polypeptide may be from any species desired, and from any natural or non-natural AAT sequence, according to the practitioner's preference. The full coding sequence of the mature human AAT gene is shown in FIG. 1. Additionally, altered AAT genes, such as genes with “silent” changes which improve expression in the host organism (“optimized” sequences), or genes encoding mutant AAT polypeptide with one or more amino acid sequence changes may also be used.

Recombinant (e.g., bacterial, such as E. coli) host cells may be engineered to produce AAT polypeptide using any convenient technology. Most commonly, a DNA sequence encoding the desired AAT polypeptide is inserted into the appropriate site in a plasmid-based expression vector which provides appropriate transcriptional and translational control sequences, although expression vectors based on bacteriophage genomic DNA are also useful. It is generally preferred that the transcriptional control sequences are inducible by a change in the environment surrounding the host cells (such as addition of a substrate or pseudosubstrate to which the transcriptional control sequences are responsive), although constitutive transcriptional control sequences are also useful. As is standard in the art, it is also preferred that the expression vector include a positive selectable marker (e.g., the β-lactamase gene, which confers resistance to ampicillin) to allow for selection against bacterial host cells which do not contain the expression vector.

The bacterial host cells are typically cultured in a liquid growth medium for production of AAT polypeptide under conditions appropriate to the host cells and expression vector. Preferably, the host cells are cultured in a bacterial fermenter to maximize production, but any convenient method of culture is acceptable (e.g., shaken flask, especially for cultures of less than a liter in volume). As will be apparent to those of skill in the art, the exact growing conditions, timing and rate of media supplementation, and addition of inducing agent (where appropriate) will vary according to the identity of the host cells and the expression construct.

After the bacterial host cells are cultured to the desired density (and after any necessary induction of expression), the cells are collected. Collection is typically conveniently effected by centrifugation of the growth medium, although any other convenient technique may be used. The collected bacterial host cells may be washed at this stage to remove traces of the growth medium, most typically by resuspension in a simple buffer followed by centrifugation (or other convenient cell collection method). At this point, the collected bacterial host cells (the “cell paste”) may be immediately processed in accordance with the invention, or it may be frozen for processing at a later time.

The cells of the cell paste are lysed to release the AAT polypeptide-containing inclusion bodies. Preferably, the cells are lysed under conditions in which the cellular debris is sufficiently disrupted that it fails to appear in the pellet under low speed centrifugation. Commonly, the cells are suspended in a buffer at about pH 5 to 9, preferably about 6 to 8, using an ionic strength of the order of about 0.01 M to 2 M preferably about 0.1-0.2 M (it is apparently undesirable to use essentially zero ionic strength). Any suitable salt, including NaCl can be used to maintain an appropriate ionic strength level. The cells, while suspended in the foregoing buffer, are then lysed by techniques commonly employed such as, for example, mechanical methods such as freeze/thaw cycling, the use of a Manton-Gaulin press, a French press, or a sonic oscillator, or by chemical or enzymatic methods such as treatment with lysozyme. It is generally desirable to perform cell lysis, and optionally bacterial cell collection, under conditions of reduced temperature (i.e., less than about 20° C.).

Inclusion bodies are collected from the lysed cell paste using any convenient technique (e.g. centrifugation), then washed. If desired, the collected inclusion bodies may be washed. Inclusion bodies are typically washed by resuspending the inclusion bodies in a wash buffer, typically the lysis buffer, preferably with a detergent added (e.g., 1% TRITON X-100 ®), then recollecting the inclusion bodies. The washed inclusion bodies are then dissolved in solubilization buffer. Solubilization buffer comprises a high concentration of a chaotroph, a pH buffer that buffers the solution to a high pH, and one or more reducing agents. The solubilization buffer may optionally contain additional agents, such as cation chelating agents and scavengers to neutralize protein-damaging free-radicals.

The instant invention utilizes urea as an exemplary chaotroph in the solubilization buffer, although guanidine hydrochloride (guanidine HCl) may also be used. Useful concentrations of urea in the solubilization buffer include about 5 M to about 8 M, for example, about 6 M, about 7 M, and about 8 M. When the chaotroph is guanidine HCl, useful concentrations include about 4 M to about 8 M, or about 5.5 M to about 6.5 M, or about 6 M.

The pH of the solubilization buffer is high, viz., in excess of pH 8.0, for example pH 9.0. Useful pH levels in the solubilization buffer are in the range of about 8.0 to about 11.0, about 9.0 to about 11.0, about 9.5 to about 10.5, about 10.0 to about 10.5, about 10.8, about 10.5 about 10, about 9.5, about 9.0, and about 8.5. As will be apparent to those of skill in the art, any pH buffering agent (or combination of agents) which effectively buffer at high pH are useful, although pH buffers which can buffer in the range of about pH 8 to about pH 9 or 10 are particularly useful. Useful pH buffering agents include tris (tris(hydroxymethyl)aminomethane), bicine(N,N-Bis(2-hydroxyethyl)glycine), HEPBS (2-Hydroxy-1,1-bis[bydroxymethyl]ethyl)amino]-1-propanesulfonic acid), TAPS ([(2-Hydroxy-1,1-bis[bydroxymethyl]ethyl)amino]-1-propanesulfonic acid), AMPD (2-Amino-2-methyl-1,3-propanediol).N-(2-Hydroxyethyl)piperazine-N′-(4-butanesulfonic acid)), and the like. The pH buffering agent is added to a concentration that provides effective pH buffering, such as from about 50 to about 150 mM, about 75 mM to about 125 mM, or about 100 mM.

Reducing agents are included in the solubilization buffer to reduce disulfide bonds and maintain cysteine residues in their reduced form. Useful reducing reagents include β-mercaptoethanol, dithiothreitol, and the like.

The solubilization buffer may contain additional components. For example, the solubilization buffer may contain a cation chelator such as a divalent cation chelator like ethylenediaminetetraacetic acid (EDTA) or ethylene glycol-bis(2-aminoethylether)-N,N,N′,N′-tetraacetic acid (EGTA). EDTA or EGTA is added to the solubilization buffer at a concentration of about 0.5 to about 5 mM, and commonly at about 1 mM. Additionally, a free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM) and other amino acids and amines.

An exemplary solubilization buffer comprises the following concentrations of the following components: about 8 M urea, about 0.1 M Tris, about 1 mM glycine, about 1 mM EDTA, about 100 mM beta-mercaptoethanol, about pH 10.5.

Another exemplary solubilization buffer comprises the following concentrations of the following components: about 8 M urea, about 0.1 M Tris, about 1 mM glycine, about 1 mM EDTA, about 100 mM beta-mercaptoethanol, pH about 8.5 to about 10.0.

The inclusion body/solubilization buffer mixture is incubated to allow full solubilization. The incubation period is generally from about six hours to about 24 hours, and more commonly about eight to about 14 hours or about 12 hours. The inclusion body/solubilization buffer mixture incubation may be carried out at reduced temperature, commonly at about 4° C. to about 10° C.

After the incubation is complete, the inclusion body/solubilization buffer mixture is clarified to remove insoluble debris. Clarification of the mixture may be accomplished by any convenient means, such as filtration (e.g., by use of depth filtration media) or by centrifugation. Clarification should be carried out at reduced temperature, such as at about 4° C. to about 10° C.

The clarified mixture is then diluted using the same or different solubilization buffer to achieve the appropriate protein concentration for refolding. Protein concentration may be determined using any convenient technique, such as Bradford assay, light absorption at 280 nm (A₂₈₀), and the like. The inventor has found that a solution having an A₂₈₀ of about 2.0 to about 10.0 (e.g., about any of 2.0, 3.0, 4.0, 5.0, 6.0, 7.0, 8.0, 9.0, and 10.0) is appropriate for use in the instant methods. If desired, this mixture may be held, refrigerated (e.g. at 4° C.), for later processing, although the mixture is not normally held for more than about four weeks. An exemplary solubilization buffer comprises the following concentrations of the following components: about 8M urea, about 0.1 M Tris, about 1 mM glycine, about 1 mM EDTA, about 10 mM beta-mercaptoethanol, about 10 mM dithiothreitol (DTT), about 1 mM reduced glutathion (GSH). The pH of the solubilization buffer for dilution may be the same as the solubilization buffer for lysing the inclusion bodies.

The concentration-adjusted inclusion body solution is first diluted with refolding buffer. The dilution is performed by adding inclusion body solution into the refolding buffer. The inclusion body solution may be diluted about 10 to about 100 fold, about 10 to about 50 fold, about 10 to about 25 fold, about 15 to about 25 fold, or about 20-fold with refolding buffer. The inclusion body solution is diluted to reduce urea and protein concentration. The final protein concentration after dilution may be about 0.01 mg/ml to about 1 mg/ml, about 0.1 mg/ml to about 0.5 mg/ml.

The refolding buffer contains a pH buffer. The refolding buffer also contains glycerol, a sugar (such as sucrose and maltose), or PEG, or any combination thereof. For example, the refolding buffer contains glycerol, sucrose, or any combination thereof. Glycerol included in the refolding buffer may have a concentration of about 5% to about 30%, about 5% to about 20%. In some embodiments, the glycerol concentration in the refolding buffer is about 10% or 30%. The refolding buffer may contain about 10% to about 30% sucrose, for example, about 25%. In some embodiments, the refolding buffer contains about 15% sucrose, about 10% glycerol and about 10% sucrose, or about 20% glycerol and about 20% sucrose. The refolding buffer may also contains polyethylene glycol (PEG), for example, with molecular weight from about 200 to about 20,000 Daltons, to help refolding and stabilizing the refolded protein. About 5% to about 10% PEG may be contained in the refolding buffer. In some embodiments, the molecular weight of the PEG is about 200 Daltons, about 300 Daltons, about 400 Daltons, or about 600 Daltons. The refolding buffer may also contain a low concentration of chaotroph, a reducing agent, and a divalent cation chelator. The refolding buffer may include additional agents, such as free-radical scavengers.

In some embodiments, the inclusion body solution is rapidly diluted with the refolding buffer. “Rapid” dilution, within the context of the invention means over a period of less than about 60 minutes. For inclusion body solution with volume of 4 liters or less, the dilution process is generally carried out during periods of less than 25 minutes, for example, about two minutes to about 25 minutes, or about five to about 20 minutes. The diluted solubilized AAT polypeptide solution is typically held for one to two hours following the completion of the rapid dilution process.

The pH of the refolding buffer may be the same or different as the solubilization buffer. The pH buffering agent in the refolding buffer may be any buffering agent or combination of buffering agents that are effective pH buffers at pH levels of about 8 to about 9.5 or about 10 or about 10.5. Useful pH buffering agents include tris (tris(hydroxymethyl)aminomethane), bicine(N,N-Bis(2-hydroxyethyl)glycine), HEPBS (2-Hydroxy-1,1-bis[bydroxymethyl]ethyl)amino]-1-propanesulfonic acid), TAPS ([(2-Hydroxy-1,1-bis[bydroxymethyl]ethyl)amino]-1-propanesulfonic acid), and AMPD (2-Amino-2-methyl-1,3-propanediol).N-(2-Hydroxyethyl)piperazine-N′-(4-butanesulfonic acid)). The pH buffering agent is added to a concentration that provides effective pH buffering, such as from about 10 to about 150 mM, about 50 to about 150 mM, about 75 mM to about 125 mM, or about 100 mM.

The divalent cation chelator may be any molecule that effectively chelates Ca⁺⁺ and other divalent cations. Exemplary cation chelators for use in the refolding buffer include ethylenediaminetetraacetic acid (EDTA) or ethylene glycol-bis(2-aminoethylether)-N,N,N′,N′-tetraacetic acid (EGTA). When EDTA or EDTA is the divalent cation chelator, it is added to the refolding buffer at a concentration of about 0.5 to about 5 mM, and commonly at about 1 mM.

The refolding buffer may further comprise a detergent, such as Tween 20, Tween 80, sodium deoxycholate, sodium cholate, and TMAO (trimethylamine-N-oxide). For example, the refolding buffer may contain Tween 20 or Tween 80 in about 0.001% to about 0.02% (e.g., about 0.005% to about 0.02%) range. In another example, the refolding buffer contains about 0.1% sodium deoxycholate, about 0.1% sodium cholate, or 0.025% TMAO.

Additional components useful in the refolding buffer include free-radical scavengers. A free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM).

An exemplary refolding buffer comprises about 20 mM Tris, about 10% glycerol or about 15% sucrose, pH about 10.5 or about 8.5. Another exemplary refolding buffer comprises about 20 mM Tris, about 5% to about 10% PEG, pH about 10.5 or about 8.5.

In some embodiments, the pH of the refolding solution is then slowly reduced from elevated pH to near neutral pH using an appropriate acid. The time period for pH reduction can range from about 20-24 hours to about 10 days, about 20 to about 50 hours, about 20 to about 40 hours, about 20 to about 30 hours, about 24 to about 40 hours. The time period for pH reduction can be at least about 20 hours, about 24 hours, about 30 hours, about 40 hours, about 48 hours, about 50 hours, about 3 days, about 4 days, about 5 days. In some embodiments, the time period for pH reduction is about 2-5 days. Appropriate acids for pH adjustment will depend on the pH buffer used in the refolding buffer. For example, when the pH buffering agent is tris, the pH should be adjusted with hydrochloric acid (HCl).

Following completion of pH adjustment, the refolding reaction is incubated for a period of about one to two hours to about 18 to 24 hours. The refolding reaction may be carried out at a room temperature (e.g., about 18-20° C.) or between about 12-18° C., depending on the preferences of the practitioner and the available facilities.

In other embodiments, the AAT polypeptide in the diluted refolding solution is incubated for a period of at least about 16 hours at a temperature of about 16° C. to about 20° C. (e.g., at room temperature) and then for about 24 hours to about 72 hours at 4° C., wherein the pH of the refolding buffer used is about 8.5 to about 10.0. The AAT polypeptide solution is then exchanged into a buffer having a pH between about 7.5 to about 8.5. This buffer may have the same formulation as the refolding buffer. Buffer exchange may be performed by dialysis or by size exclusion chromatography. A concentration step (e.g., by ultrafiltration) may be performed before the buffer exchange step. In some embodiments, the solution is incubated for 16 hours at 20° C., and the pH of the refolding buffer is 8.5. The refolded AAT polypeptide may be kept at 4° C. for 2-7 days before proceeding for purification.

Following the refolding reaction, properly refolded AAT polypeptide may be concentrated and further purified. Concentration of the refolded protein may be accomplished using any convenient technique, such as ultrafiltration, diafilitration, chromatography (e.g., ion-exchange, hydrophobic interaction, or affinity chromatography) and the like. Where practical, it is preferred that concentration be carried out at reduced temperature (e.g., about 4-10° C.).

Any convenient protein purification protocol may be used. In some embodiments, two types of chromatography may be used for purification. For example, size exclusion chromatography (SEC) and/or ion exchange chromatography may be used.

Size exclusion chromatography (SEC) may be performed using any convenient chromatography medium which separates properly folded AAT polypeptide from unfolded AAT polypeptide and multimeric AAT polypeptide. The inventors have found that media having the ability to size fractionate proteins of about 10⁴ to about 6×10⁵ daltons (globular proteins) are useful for this step. Exemplary SEC media include Sephacryl® 300 and Superdex 75. This step may also be used to perform buffer exchange, if so desired. The exact conditions for SEC will depend on the exact chromatography media selected, whether buffer exchange is to be accomplished, the requirements of any later purification steps, and other factors known to those of skill in the art.

The properly folded AAT polypeptide may be further purified utilizing ion exchange chromatography, for example, HiTrapQ XL anion exchange column shown in Example 3.

The properly folded AAT polypeptide may be further separated from improperly folded or unfolded AAT polypeptide using a hydrophobic interaction chromatography resin (e.g., phenyl sepharose chromatography, butyl sepharose chromatography, octyl sepharose chromatography) in the presence of a salt. Under appropriate concentration of salt (e.g., about 0.25 to about 1.2 M of (NH₄)₂SO₄, or about 1.5 M to about 3.5 M of NaCl), the improperly folded or unfolded AAT polypeptide binds to the resin while properly folded AAT polypeptide flows through the resin. For example, NaCl or (NH₄)₂SO₄ may be used. In one embodiment, AAT polypeptide in 20 mM Tris, 1 M (NH₄)₂SO₄, 7.5% sucrose, 0.005% Tween 20, 1 mM DTT, pH 7.6 is loaded onto a phenyl sepharose column, and the flow-through faction containing the properly folded AAT polypeptide is collected.

The refolded AAT polypeptides may be modified to increase their half lives in an individual, such as a human. For example, the AAT polypeptide may be pegylated to reduce systemic clearance with minimal loss of biological activity. Any pegylation methods known in the art may be used. See, e.g., Cantin et al., Am. J. Respir. Cell Mol. Biol. 27:659-665, 2002; Travis et al., Methods Enzymml. 80:754-766, 1981; Reberts et al., Advanced Drug Delivery Reviews 54:459-476, 2002. One pegylation method is described in detail in Example 4. In some embodiments, the AAT polypeptide is pegylated with a PEG having molecular weight of about 20 to about 40 kD. In some embodiments, the PEG have a molecular weight of about 21 kD. In some embodiments, the PEG molecule is conjugated to Cys²³² of the AAT polypeptide, wherein the amino acid numbering for Cys²³² is based on the numbering in SEQ ID NO:1. The half life of the modified polypeptides may be increased at least about 10%, at least about 20%, at least about 30%, at least about 50%, at least about 100%, at least about 2 fold, at least about 5 fold as compared to the unmodified AAT polypeptide.

Biological activity of AAT polypeptide produced from the properly folded recombinant AAT polypeptide produced in accordance with the invention may be measured using any acceptable assay method known in the art. See, e.g., Cantin et al., Am. J. Respir. Cell Mol. Biol. 27:659-665, 2002; Travis et al., Methods Enzymml. 80:754-766, 1981. An exemplary method of measuring AAT polypeptide activity is described herein in Example 3, which measures inhibition of human leukocyte elastase (HLE) and porcine pancreatic elastase activity in vitro using artificial substrate. Other assays include administering AAT polypeptide and HLE into animals and measuring lung anti-HLE protection by the AAT polypeptide. Cantin et al., Am. J. Respir. Cell Mol. Biol. 27:659-665, 2002. For example, mice may be instilled intranasally with AAT polypeptide, and subsequently instilled with HLE. Hemoglobin content is determined as an index of HLE-mediated lung injury. Cantin et al., Am. J. Respir. Crit. Care Med. 157:464-469, 1998.

As is well understood in the art, all concentrations and pH values need not be exact and reference to a given value reflects standard usage in the art, does not mean that the value cannot vary.

C. Pharmaceutical Compositions and Kits

The invention provides pharmaceutical compositions comprising an AAT polypeptide and a pharmaceutical acceptable excipient. The AAT polypeptide may be in the form of lyophilized formulations or aqueous solutions. Acceptable excipients are nontoxic to recipients at the dosages and concentrations employed, and may comprises buffers such as phosphate, citrate; salts such as sodium chloride; sugars such as sucrose; and/or polyethylene glycol (PEG). See Remington: The Science and Practice of Pharmacy 20th Ed. (2000) Lippincott Williams and Wilkins, Ed. K. E. Hoover. The AAT polypeptide may be formulated for different routes of drug delivery formulations, such as liquid or lyophilized formulation for intravenous (IV) injection, and dry poerder formulation or aerosolization formulation for deep lung delivery. These formulations are known in the art. See, e.g., Drug Delivery to the Lung, Bisgaard H., O'Callaghan C and Smaldone G C, editors, New York; Marcel Dekker, 2002.

The AAT polypeptide may be produced by any methods described herein. In some embodiments, the AAT polypeptide is produced from bacterial (e.g., E. coli) inclusion bodies. In some embodiments, the AAT polypeptide is unglycosylated. In some embodiments, the AAT polypeptide has a purity of at least about any of 80%, 85%, 90%, 95%, 96%, 97%, 98%, and 99%. In some embodiments, the specific activity of the AAT polypeptide (e.g., as determined by porcine pancreatic elastase inhibition) is no less than about any of 0.3, 0.35, 0.4, 0.45, 0.5, 0.55, 0.6, 0.65, 0.7, 0.75, 0.8, 0.85, 0.9, and 0.95 mg active AAT polypeptide per milligram of total protein.

The invention also provides kits comprising an AAT polypeptide described herein. Kits of the invention include one or more containers comprising an AAT polypeptide. The containers may be vials, bottles, jars, or flexible packaging. The container may contain unit dosage or sub-unit dosage. For example, the AAT polypeptide may be packaged in single use vials, each containing either 500 mg or 1,000 mg active AAT polypeptide. The container may have a sterile access port (e.g., a stopper pierceable by a hypodermic injection needle). At least one active agent in the container is an AAT polypeptide. The container may further comprise a second pharmaceutical active agent.

The kits may further comprise additional components, such as a second container containing sterile water for reconstitution of lyophilized AAT polypeptide. The kits may further comprise instructions for using the AAT polypeptide for therapeutic purposes, e.g., for treating conditions associated with deficiency of AAT. The instructions generally include information as to dosage, dosing schedule, and route of administration for the intended treatment. The instructions are typically written instructions on a label or package insert (e.g., a paper sheet included in the kit), but machine-readable instructions (e.g., instructions carried on a magnetic or optical storage disk) are also acceptable. The kits may also include devices for dry powder or aerosole delivery to the lung.

The following examples are provided to illustrate, but not to limit, the invention.

### EXAMPLES

### Example 1

### Refolding of Recombinant AAT Polypeptides using pH Reducing Method

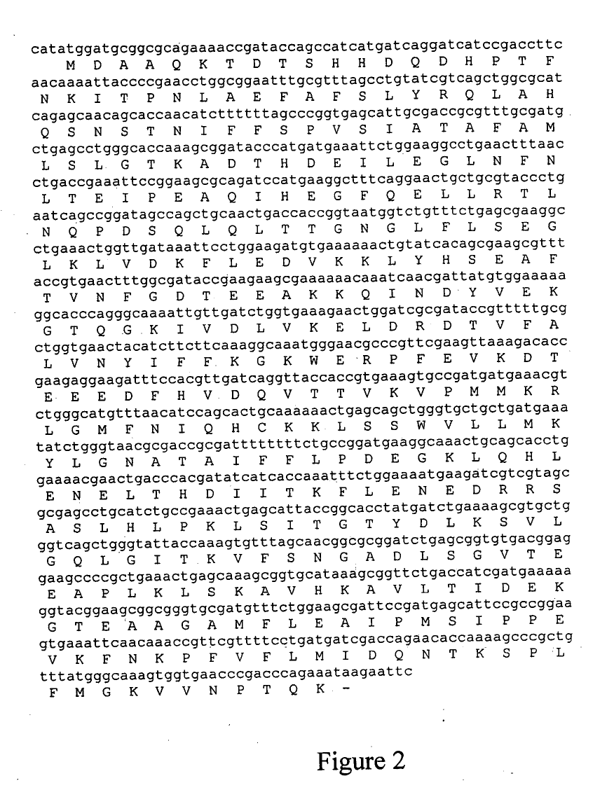

Vector construction and expression. A DNA fragment (SEQ ID NO:4) encoding human Δ5 AAT polypeptide (SEQ ID NO:3) as shown in FIG. 2 was produced by PCR amplification. Δ5 AAT polypeptide lacks amino acids 1-5 of SEQ ID NO:1 shown in FIG. 1 and has an artificial methionine start site which facilitates expression in E. coli. The polynucleotide sequence in the DNA fragment encoding Δ5 AAT polypeptide was optimized for expression in E. coli. The PCR product was inserted into the Nde 1 and EcoR1 sites of pET11 (Novegen, San Diego, Calif.) modified to include multiple cloning sites. After PCR, ligation, and transformation into the BL21 (DE3) strain of E. coli, single colonies were selected and amplified and then ultimately the construct was sequenced to assure the correctness of the DNA sequence.

Constructs for expression of full-length human AAT using DNA fragment (SEQ ID NO:2) or a DNA fragment encoding human Δ10 AAT polypeptide which lacks amino acids 1-10 of SEQ ID NO:1 but possesses an artificial methionine for initiation of the translation were produced as described above.

To produce AAT polypeptides, AAT polypeptide expression vectors were transfected into BL21 (DE3) strain of E. coli and plated on ZB plates with ampicillin. A single colony was selected and used to inoculate 100 mL of ZB media (10 g/l NZ amine A (Sigma) and 5 g/l NaCl) with ampicillin and grown overnight (approximately 16 hours) at 30° C. The 20 mL of the 100 mL starter culture was then used to inoculate 1 L of LB media with ampicillin, and the culture was incubated at 37° C. with shaking until the optical density at 600 nm (OD₆₀₀) reached 0.4-0.6. Isopropyl-beta-D-thiogalactopyranoside (IPTG) was then added to 0.5 mM to induce AAT polypeptide expression, and the culture was incubated a further three hours with shaking. Large scale expression was accomplished utilizing multiple IL shaker flasks at 37° C.

Expression of full-length wild type gene coded human AAT resulted in very low level of expression. Both Δ5 AAT polypeptide and Δ10 AAT polypeptide were expressed in bacterial cells with acceptable yields.

Processing of inclusion bodies before refolding: The inclusion bodies were harvested from bacteria. Bacterial cells were collected by centrifugation, then resuspended in 20 mL of TN (250 mM NaCl, 100 mM Tris, pH 8.0) with 1% TRITON X-100 ®. Ten milligrams of lysosyme were added, and the cell suspension was frozen at −20° C. overnight. The lysate was then thawed and 20 μL of 1 M magnesium sulfate and 100 μg of DNase were added. The cells were incubated, with stirring, until the released bacterial DNA was completely dissolved. The lysate was then diluted with 250 mL of TN with 1% TRITON X-100 ® and the mixture was stirred for 2-4 hours. Inclusion bodies were collected by centrifugation, and washed three times (by resuspension and centrifugation).

Refolding of AAT polypeptide. The washed inclusion bodies were dissolved in the solubilization buffer having 8M urea, 0.1 M Tris, 1 mM glycine, 1 mM EDTA, 100 mM beta-mercaptoethanol, pH 10.5 at high OD280 (20-40), and stirred gently for about 12 hours at 4° C. The solubilized material was then spun in a Beckman 70 Ti rotor at 30 Krpm for 30 minutes to remove insoluble debris. The absorbance at 280 nm (OD280) of the solubilized inclusion body solution was adjusted to 2.0 with 8M urea, 0.1 M Tris, 1 mM glycine, 1 mM EDTA, 10 mM beta-mercaptoethanol, 10 mM dithiothreitol (DTT), 1 mM reduced glutathion (GSH), pH 10.5.

The clarified solution was rapidly diluted into 20 volumes of a refolding buffer containing 20 mM Tris, 10% glycerol, pH 10.5, with a final A₂₈₀ to 0.1. The resulting solution was adjusted to pH 7.6 with 1 M HCl stepwise over 2-4 days. Refolded AAT polypeptide was generated.

Using higher concentration of glycerol (20%) in the refolding buffer, or replacing glycerol in the refolding buffer with 20% sucrose or a combination of 10% sucrose and 10% glycerol was tested. In some experiments, Tween 20 (0.005%-0.01%) was also included in the refolding buffer. All these conditions generated properly folded (active) AAT polypeptide.

### Example 2

### Refolding of Recombinant AAT Polypeptides using Static pH

The inclusion bodies with the expressed AAT polypeptides were refolded using static pH conditions. The washed inclusion bodies were dissolved in the solubilization buffer having 8M urea, 0.1 M Tris, 1 mM glycine, 1 mM EDTA, 100 mM beta-mercaptoethanol, pH 10.5 at high OD280 (20-40), and stirred gently for about 12 hours at 4° C. The solubilized material was then spun in a Beckman 70 Ti rotor at 30 Krpm for 30 minutes to remove insoluble debris. The absorbance at 280 nm (OD280) of the solubilized inclusion body solution was adjusted to 2.0 with 8M urea, 0.1 M Tris, 1 mM glycine, 1 mM EDTA, 10 mM beta-mercaptoethanol, 10 mM dithiothreitol (DTT), 1 mM reduced glutathion (GSH), pH 10.5. The clarified solution was rapidly diluted into 20 volumes of a refolding buffer of 20 mM Tris, 10% glycerol, pH 8.5, with a final A₂₈₀ to 0.1. The solution was kept at 20° C. for 16 hours, and then kept at 4° C. for 2-7 days before proceeding for concentration, buffer exchange, and purification as described in Example 3.

### Example 3

### Purification of Refolded Recombinant AAT Polypeptides and Determination of Biological Activity

Refolded Δ5 AAT polypeptide or Δ10 AAT polypeptide as described in Example 1 was concentrated to an A₂₈₀>20.0 using a Pellicon device from Millipore and then centrifuged at 30,000 rpm in a Type 70 Ti rotor using a Beckman LE-80K Ultracentrifuge for 30 minutes to remove extraneous insoluble debris. The recovered supernatant was then loaded onto a 5.0×90 cm Superdex 75 (Amersham) size exclusion column pre-equilibrated with 20 mM Tris, 0.2 M NaCl, 15% sucrose, 0.005% Tween 20, 1 mM DTT, pH 7.6 to separate monomeric Δ5 AAT polypeptide or Δ10 AAT polypeptide from unfolded, aggregated, or multimeric forms of the polypeptides. 10 mL fractions that were collected off the column were analyzed by A₂₈₀ and SDS PAGE. Monomeric partially purified fractions were then pooled and loaded onto a HiTrap Q XL anion exchange column (Amersham) and then eluted against a 0-1000 mM NaCl gradient in a 20 mM MES buffer pH 6.2 containing 1 mM DTT on a Pharmacia AKTA FPLC. The anion exchange chromatography fractions were analyzed by SDS PAGE under non-reducing conditions; highly purified fractions were combined. The concentration of the pooled material was determined by molar extinction in 6M guanidine, 20 mM NaPi, pH=6.5 using a computed extinction coefficient ε₂₈₀=19060 M⁻¹ cm⁻¹ for the Δ5 and Δ10 AAT polypeptides.

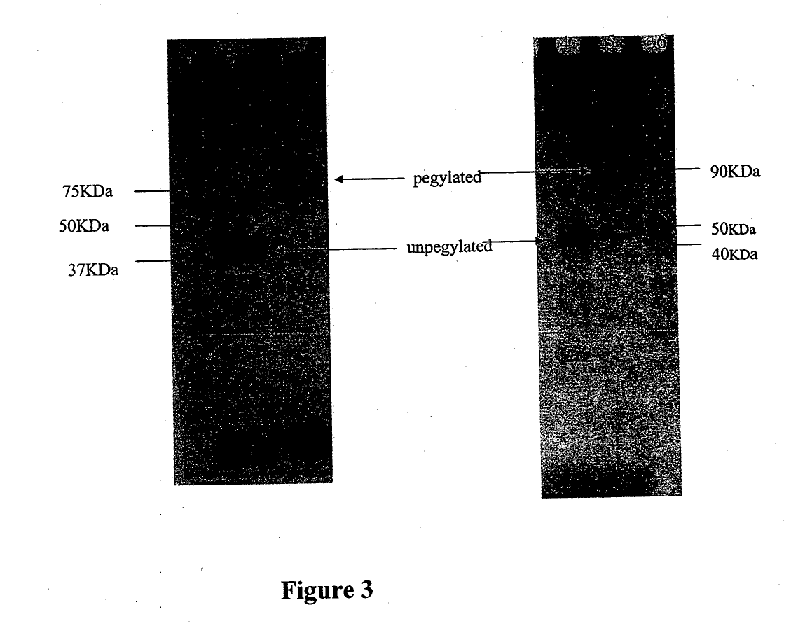

As shown in FIG. 3, a monomeric Δ5 AAT polypeptide and Δ10 AAT polypeptide (refolded with 10% glycerol in the refolding buffer) in near homogeneity were produced and purified.

Inhibitory properties of the refolded and purified Δ5 AAT polypeptide and Δ10 AAT polypeptide in blocking human leukocyte elastase (HLE) and porcine pancreatic elastase (PPE) were tested and compared to commercially obtained glycosylated full length AAT isolated from human plasma and purchased from Calbiochem (San Diego, Calif. Cat. #178251 ). The PPE isolated from hog pancreas was purchased from Sigma-Aldrich (St. Louis, Mo., cat. #E7885); and the HLE isolated from human sputum was purchased from Molecular Innovations (Southfield, Mich. Cat#HNE). A range of concentrations 0.3 nM to 14 nM of human Δ5 AAT polypeptide or commercially available full length glycosylated plasma AAT were incubated with a fixed concentration 1.4 nM of either HLE or PPE for 15 minutes at 37° C. and then aliquots of the incubate were mixed with 1 mM of the elastase substrates N-succinyl-ala-ala-ala-p-nitroanilide (chromogenic substrate for PPE) or N-methoxy-succinyl-ala-ala-pro-val-p-nitroanilide (chromogenic substrate for HLE). The kinetics of hydrolysis of the substrate was monitored at 21° C. at 405 nm using a Molecular Devices Spectrophotometer (Spectramax Plus). The initial velocity of each reaction was determined and the percentage activity relative to a control (no AAT or AAT polypeptides) was determined. The percent elastase activity was plotted against the stoichiometric molar ratio of concentrations of AAT polypeptide:elastase used in the corresponding reaction. The precise concentrations of stocks of each form of AAT polypeptides, PPE, and HLE used in the experiment were determined prior to the reactions using each respective polypeptide or protein's known extinction coefficient as obtained using the computer software program ProtParam from the ExPASY proteomics server at the Swiss Institute of Bioinformatics (http://www.expasy.ch).

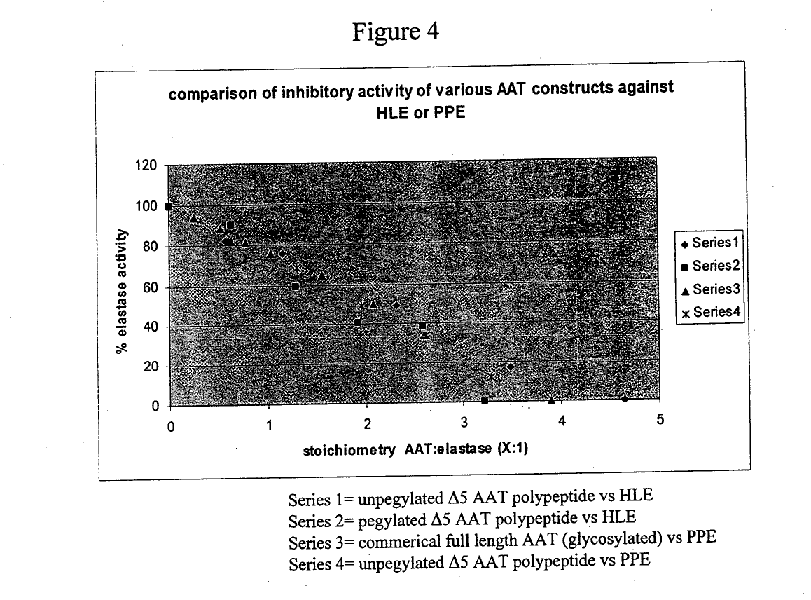

As shown in FIG. 4, Δ5 AAT polypeptide refolded as described in Example 1 demonstrated inhibitory activity against HLE and PPE, and the inhibitory activity was comparable to commercially obtained human plasma AAT. Δ5 AAT polypeptide refolded as described in Example 2, and Δ10 AAT polypeptide refolded as described in Examples 1 and 2 also demonstrated blocking activity to HLE and PPE.

### Example 4

### Pegylation of Refolded Recombinant AAT Polypeptide

Δ5 AAT polypeptide refolded and purified as described in Examples 1 and 3 in 20 mM MES 6.2,200 mM NaCl, 1 mM DTT was exchanged over a PD-10 (BioRad) column pre-equilibrated with 50 mM NaPi pH 7.5, 200 mM NaCl to remove DTT and change the pH to 7.5 according to the manufacturer's protocol. The buffer-exchange process was usually performed twice to be absolutely certain there were no trace levels of DTT present because this reducing agent interferes with the pegylation reaction. The buffer-exchanged Δ5 AAT polypeptide was quantitated by molar extinction. Solid PEG-mal20 (polyethylene glycol maleimide 20 having approximate molecular weight of 21 KDa, Nektar, Huntsville, Ala.) stored at −20° C. under argon gas was added to the solution of Δ5 AAT polypeptide at a molar ratio of 5:1 to 10:1 and incubated at 37° C. for 30 minutes. The reaction was stopped by adding 20 mM DTT and incubating for an additional 5 minutes at 37° C. The reaction mixture was diluted at least 4× with distilled water to dilute the salt concentration to below 50 mM NaCl and was then loaded to a HiTrap Q anion exchange column to separate pegylated from unpegylated AAT polypeptide. Pegylated AAT polypeptide has a lower affinity to the HiTrap Q anion exchange resin than unpegylated AAT polypeptide. These two forms of AAT were selectively separated in different salt gradient fractions during elution.

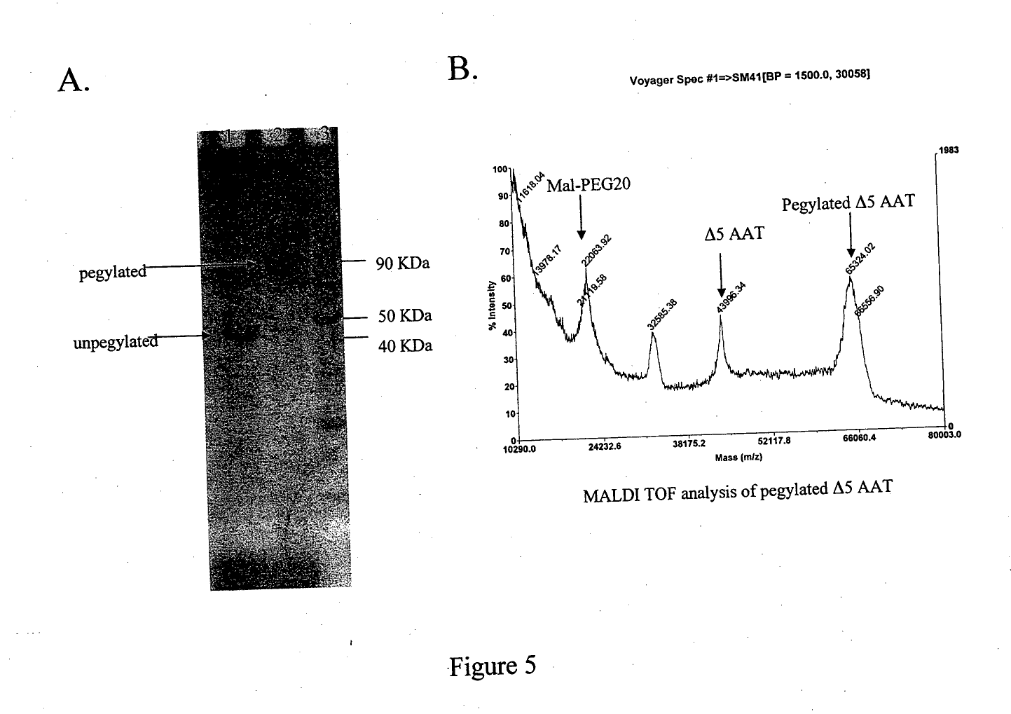

The success of the pegylation reaction was determined using SDS-PAGE (FIG. 5A) and MALDI-TOF mass spectrometry (FIG. 5B). Inhibitory activity of the pegylated Δ5 AAT polypeptide in blocking HLE or PPE enzymatic activity is shown in FIG. 4. As shown in FIGS. 4 and 5, Δ5 AAT polypeptide was successfully pegylated, and functional properties of the pegylated Δ5 AAT polypeptide in inhibiting HLE and PPE were equivalent to non-pegylated Δ5 AAT polypeptide in vitro. Δ10 AAT was also successfully pegylated (FIG. 3) and was shown to retain elastase inhibitory activity.

### Example 5

### Purification of Properly Folded AAT Polypeptide from Improperly Folded or Unfolded AAT Polypeptide by Phenyl Sepharose Chromatography

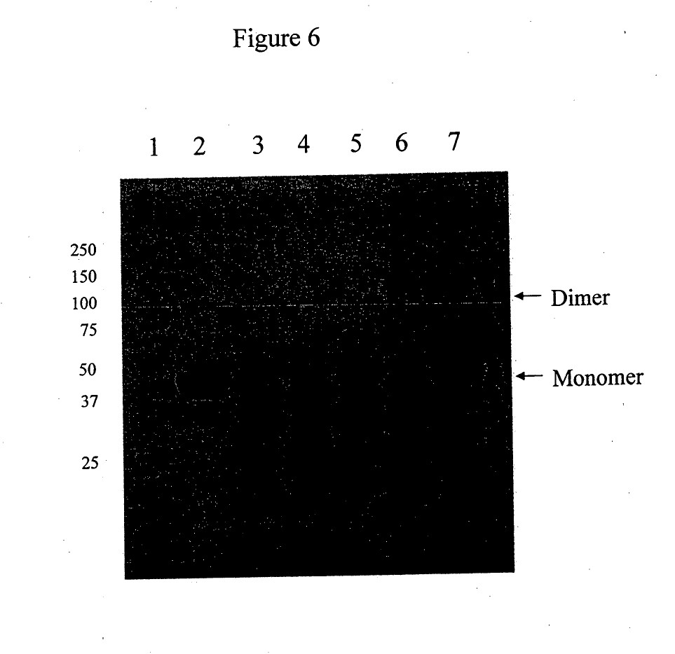

The refolded AAT polypeptide purified using methods described above was further purified by hydrophobic interaction chromatography (HIC), such as phenyl Sepharose chromatography. A phenyl Sepharose column (Amersham Biosciences) was equilibrated with buffer containing: 20 mM Tris, 1 M (NH₄)₂SO₄, 10% sucrose, 0.005% Tween 20, 1 mM DTT, pH 7.6. Refolded Δ5 AAT polypeptide, which was purified by size exclusion column and anion exchange column in Example 3, was further purified using the phenyl Sepharose column. Refolded Δ5 AAT polypeptide in the same buffer as used for equilibrating the phenyl Sepharose column was applied to the column. Improperly folded or unfolded Δ5 AAT polypeptide bound to the column, and the properly folded Δ5 AAT flowed through and was collected and concentrated. The column was eluted with an elution buffer containing 20 mM Tris, 7.5% sucrose, 0.005% Tween 20, 1 mM DDT, pH 7.6 to collect the improperly folded or unfolded Δ5 AAT polypeptide. Fractions of Δ5 AAT polypeptide collected from the hydrophobic interaction chromatography were run on SDS-PAGE (FIG. 6). The Δ5 AAT polypeptide purified through the phenyl Sepharose column was tested for activity, and its activity was compared to Zemaira α1-proteinase inhibitor (human) from Aventis Behring L.L.C.

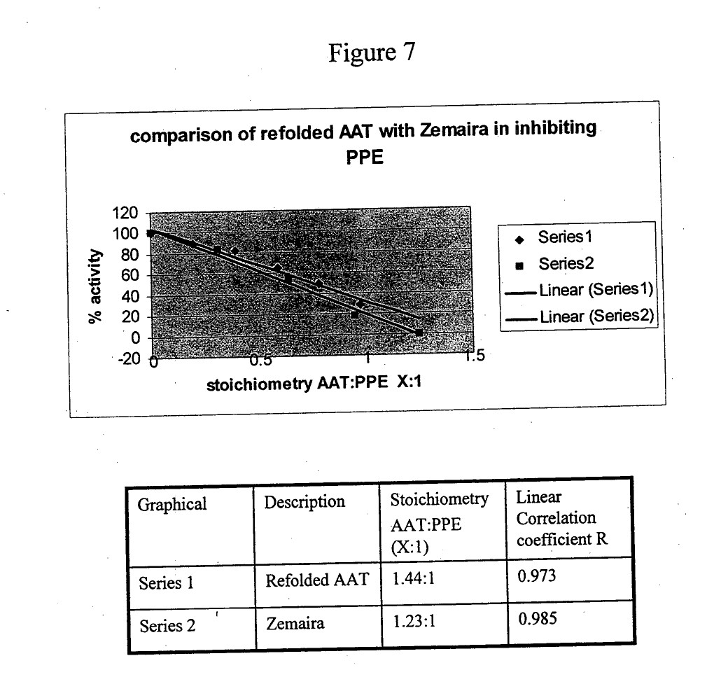

Purified Δ5 AAT polypeptide was diluted to 250 μg/μl using reaction buffer: 20 mM Tris, 38 mM NaCl, 0.01% Tween 20, pH 8.8. The PPE (Porcine Pancreatic Elastase) was prepared in the reaction buffer, and different concentration of Δ5 AAT polypeptide or Zemaira® α1-proteinase inhibitor was incubated with the PPE at 37° C. for 15 min. The reaction mix was further diluted with H₂O and then substrate P-ala-ala-ala substrate (Sigma) was added to the mix to initiate the PPE hydrolysis reaction. The reaction was monitored at 405 nm in a 96-well plate. The inhibitory activity of the PPE in the presence of various concentration (as plotted as the stoichiometry of AAT:PPE) of refolded Δ5 AAT polypeptide and Zemaira is shown in FIG. 7. Data in FIG. 7 indicated that the specific activity of the refolded and purified Δ5 AAT polypeptide was similar to Zemaira®.

### Example 6

### Refolding of Recombinant AAT Polypeptide using a Refolding Buffer Containing PEG, Sucrose or a Detergent

Refolding buffers containing PEG, sucrose, or a detergent were tested. Refolding of Δ5 AAT polypeptide was performed as described in Example 1, except the refolding buffer contains 20 mM Tris, pH 10.5, and any of 1) 1-10% PEG (PEG200, PEG300, PEG400, PEG600, PEG1000, or PEG3000, all from Sigma, U.S.A.); 2) 10-20% sucrose; and 3) a detergent (Sodium Lauroyl Sarcosine (from Arresco), TMAO (trimethylamine-N-oxide, from Sigma), sodium deoxycholate (NaDeCholate, from Sigma), sodium cholate (NaCholate, from Sigma), CTAB (cetyltrimethylammonium bromide, from Sigma), beta-cyclodextrin (from Sigma), or Pluronic F-68 (from Sigma). By testing the activity of refolded Δ5 AAT polypeptide in blocking human leukocyte elastase or porcine pancreatic elastase, the highest activity Δ5 AAT polypeptide was obtained with 10% PEG200 or 5% PEG600 in the refolding buffer among all the PEGs tested. Among the detergents tested, NaDeCholate (0.1%), NaCholate (0.1%), or TMAO (0.025%) facilitate refolding of Δ5 AAT polypeptide, but to a lesser extent than PEG.

Although the foregoing invention has been described in some detail by way of illustration and example for purposes of clarity and understanding, it will be apparent to those skilled in the art that certain changes and modifications may be practiced. Therefore, descriptions and examples should not be construed as limiting the scope of the invention, which is delineated by the appended claims.

The claimed invention is:

1. A method for producing a refolded recombinant AAT polypeptide comprising:
a) solubilizing a denatured AAT polypeptide with a solubilization buffer comprising a high concentration of chaotroph, a reducing agent, and having a pH of about 8.5 to about 11.0, to produce a solubilized AAT polypeptide solution;
b) diluting the solubilized AAT polypeptide solution with a refolding buffer by adding the solubilized AAT polypeptide solution into the refolding buffer to produce a diluted solubilized AAT polypeptide solution, wherein the refolding buffer comprises glycerol, a sugar, or polyethylene glycol (PEG), or any combination thereof; and
c) reducing the pH of the diluted solubilized AAT polypeptide solution to a pH of about 7.5 to about 8.5, wherein said pH reducing is carried out over a period of at least about 20 hours, thereby producing a refolded AAT polypeptide.

2. The method of claim 1, wherein the AAT polypeptide comprises amino acid sequence of SEQ ID NO:1.

3. The method of claim 2, wherein the AAT polypeptide has one or more amino acid residues deleted within amino acid residues 1-15 of SEQ ID NO:1.

4. The method of claim 1, wherein the AAT polypeptide comprises the amino acid sequence selected from the group consisting of 2-394, 3-394, 4-394, 5-394, 6-394, 7-394, 8-394, 9-394, 10-394, and 11-294 of SEQ ID NO:1.

5. The method of claim 1, wherein the AAT polypeptide comprises the amino acid sequence of SEQ ID NO:3.

6. The method of claim 1, wherein the chaotroph is urea or guanidine hydrochloride.

7. The method of claim 1, wherein the refolding buffer comprises about 5% to about 30% glycerol.

8. The method of claim 1, wherein the refolding buffer comprises about 10% to about 30% sucrose.

9. The method of claim 1, wherein the refolding buffer comprises PEG having molecular weight about 200 to about 20,000 Daltons.

10. The method of claim 1, wherein the refolding buffer further comprises a detergent selected from the group consisting of Tween 20, Tween 80, sodium deoxycholate, sodium cholate, and trimethylamine-N-oxide (TMSO).

11. The method of claim 1, wherein the solubilization buffer and the refolding buffer have the same pH.

12. The method of claim 1, wherein the solubilization buffer comprises about 8 M urea, about 0.1 M Tris, about 1 mM glycine, about 1 mM EDTA, about 100 mM β-mercaptoethanol, at about pH 10.0 to about pH 10.8.

13. The method of claim 1, further comprising adjusting the A₂₈₀ of the solubilized AAT polypeptide solution to about 2.0 to about 10.0 with a solubilization buffer before diluting the solubilized AAT polypeptide solution with the refolding buffer.

14. The method of claim 13, wherein the solubilization buffer comprises about 8 M urea, about 0.1 M Tris, about 1 mM glycine, about 1 mM EDTA, about 10 mM β-mercaptoethanol, about 10 mM dithiothreitol (DTT), about 1 mM reduced glutathione (GSH), at about pH 10.0 to about pH 10.8.

15. The method of claim 1, wherein the solubilized AAT polypeptide is diluted about twenty-fold into the refolding buffer.

16. The method of claim 1, wherein the refolding buffer comprises about 20 mM Tris, pH about 10.5, and any of 1) about 10% to about 30% glycerol, 2) about 10 to about 30% sucrose, 3) about 20% glycerol and about 20% sucrose, 4) about 10% glycerol and about 10% sucrose, and 5) about 5% to about 10% PEG.

17. The method of claim 1, wherein the refolding buffer further comprises about 0.001% to about 0.02% Tween 20.

18. The method of claim 1, wherein the denatured AAT polypeptide is from bacterial inclusion bodies.

19. The method of claim 1, further comprising purifying the refolded AAT polypeptide.

20. A method for producing a refolded recombinant AAT polypeptide comprising:
a) solubilizing a denatured AAT polypeptide with a solubilization buffer comprising a high concentration of chaotroph, a reducing agent, and having a pH of about 8.5 to about 10.5, to produce a solubilized AAT polypeptide solution;
b) diluting the solubilized AAT polypeptide solution with a refolding buffer having a pH of about 8.5 to about 10.5 by adding the solubilized AAT polypeptide solution into the refolding buffer to produce a diluted solubilized AAT polypeptide solution, wherein the refolding buffer comprises glycerol, a sugar, or PEG, or any combination thereof;
c) incubating the diluted solubilized AAT polypeptide solution for at least about 16 hours at a temperature of about 16° C. to about 20° C.;
d) further incubating the diluted solubilized AAT polypeptide solution at about 4° C. for about 24 to about 72 hours; and
e) exchanging the diluted solubilized AAT polypeptide solution to a buffer having a pH of about 7.5 to about 8.5, thereby producing a refolded AAT polypeptide.

21. The method of claim 20, the solubilization buffer and the refolding buffer have the same pH.

22. The method of claim 20, further comprises a step of concentrating the diluted solubilized AAT polypeptide solution before step e).

23. The method of claim 20, wherein step e) is performed by dialysis or size exclusion chromatography.

24. The method of claim 20, wherein the solubilization buffer comprises about 8 M urea, about 0.1 M Tris, about 1 mM glycine, about 1 mM EDTA, about 100 mM β-mercaptoethanol, at about pH 8.5 to about pH 10.5.

25. The method of claim 20, further comprising adjusting the A₂₈₀ of the solubilized AAT polypeptide solution to about 2.0 to about 5.0 with a solubilization buffer before diluting the solubilized AAT polypeptide solution with the refolding buffer.

26. The method of claim 25, wherein the solubilization buffer comprises about 8 M urea, about 0.1 M Tris, about 1 mM glycine, about 1 mM EDTA, about 10 mM β-mercaptoethanol, about 10 mM dithiothreitol (DTT), about 1 mM reduced glutathione (GSH), at about pH 8.5 to about pH 10.5.

27. The method of claim 20, wherein the solubilized AAT polypeptide is diluted about twenty-fold into the refolding buffer.

28. The method of claim 20, wherein the refolding buffer comprises about 20 mM Tris, pH about 10.5, and any of 1) about 10% to about 30% glycerol, 2) about 10 to about 30% sucrose, 3) about 20% glycerol and about 20% sucrose, 4) about 10% glycerol and about 10% sucrose, and 5) about 5% to about 10% PEG.

29. The method of claim 20, wherein the refolding buffer further comprises about 0.005% to about 0.02% Tween 20.

30. A method for purification of a properly folded AAT polypeptide from improperly folded or unfolded AAT comprising:
a) binding of. the improperly folded or unfolded AAT polypeptide to a hydrophobic interaction chromatography resin in the presence of a salt; and
b) collecting the properly folded AAT polypeptide which is not bound to the resin.

31. The method of claim 30, wherein the salt is (NH₄)₂SO₄ or NaCl.

32. The method of claim 31, wherein about 0.25 M to about 1.2 M (NH₄)₂SO₄ or about 1.0 M to about 3.5 M NaCl is used.

33. A composition comprising an unglycosylated AAT polypeptide produced by the method of claim 1 or claim 18.

34. The composition of claim 33, further comprising a pharmaceutically acceptable excipient.

35. The composition of claim 33, wherein the AAT polypeptide is conjugated to a polyethylene glycol (PEG) molecule.

36. A kit for treating AAT deficiency comprising an unglycosylated AAT polypeptide produced by the method of claim 1 or claim 18.


SEQUENCE LISTING (4 sequences)

SEQ ID NO: 1 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIDQNTKSPLFMGKVVNPTQK

SEQ ID NO: 2 - DNA, 1185 bp, Homo sapiens
gaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcttgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaccaaaataccaagtctcccctcttcatgggaaaagtggtgaatcccacccaaaaataa

SEQ ID NO: 3 - protein, 390 aa, Artificial Sequence
  Synthetic Construct
MDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIDQNTKSPLFMGKVVNPTQK

SEQ ID NO: 4 - DNA, 1182 bp, Artificial Sequence
  Synthetic Construct
catatggatgcggcgcagaaaaccgataccagccatcatgatcaggatcatccgaccttcaacaaaattaccccgaacctggcggaatttgcgtttagcctgtatcgtcagctggcgcatcagagcaacagcaccaacatcttttttagcccggtgagcattgcgaccgcgtttgcgatgctgagcctgggcaccaaagcggatacccatgatgaaattctggaaggcctgaactttaacctgaccgaaattccggaagcgcagatccatgaaggctttcaggaactgctgcgtaccctgaatcagccggatagccagctgcaactgaccaccggtaatggtctgtttctgagcgaaggcctgaaactggttgataaattcctggaagatgtgaaaaaactgtatcacagcgaagcgtttaccgtgaactttggcgataccgaagaagcgaaaaaacaaatcaacgattatgtggaaaaaggcacccagggcaaaattgttgatctggtgaaagaactggatcgcgataccgtttttgcgctggtgaactacatcttcttcaaaggcaaatgggaacgcccgttcgaagttaaagacaccgaagaggaagatttccacgttgatcaggttaccaccgtgaaagtgccgatgatgaaacgtctgggcatgtttaacatccagcactgcaaaaaactgagcagctgggtgctgctgatgaaatatctgggtaacgcgaccgcgattttttttctgccggatgaaggcaaactgcagcacctggaaaacgaactgacccacgatatcatcaccaaatttctggaaaatgaagatcgtcgtagcgcgagcctgcatctgccgaaactgagcattaccggcacctatgatctgaaaagcgtgctgggtcagctgggtattaccaaagtgtttagcaacggcgcggatctgagcggtgtgacggaggaagccccgctgaaactgagcaaagcggtgcataaagcggttctgaccatcgatgaaaaaggtacggaagcggcgggtgcgatgtttctggaagcgattccgatgagcattccgccggaagtgaaattcaacaaaccgttcgttttcctgatgatcgaccagaacaccaaaagcccgctgtttatgggcaaagtggtgaacccgacccagaaataagaattc